(19) United States
(12) Patent Application Publication
(10) Pub. No.: US 2024/0059627 A1
(43) Pub. Date: Feb. 22, 2024

(54) PROCESSES FOR PRODUCING MIXTURES OF DIFFERENT OLEFINS

(71) Applicant: Gevo, Inc., Englewood, CO (US)

(72) Inventors: Jonathan Smith, Highlands Ranch, CO (US); Andrew Ingram, Denver, CO (US)

(21) Appl. No.: 18/450,562

(22) Filed: Aug. 16, 2023

Related U.S. Application Data
(60) Provisional application No. 63/398,802, filed on Aug. 17, 2022.
(60) Provisional application No. 63/404,465, filed on Sep. 7, 2022.

(51) Int. Cl. C07C 1/207; C07D 307/33; B01J 23/06; B01J 29/40; B01J 27/14; B01J 21/02; B01J 35/00

(52) U.S. Cl. CPC C07C 1/2078; C07D 307/33; B01J 23/06; B01J 29/40; B01J 27/14; B01J 21/02; B01J 35/0066; C07C 2529/06; C07C 2523/06; C07C 2521/06

(57) ABSTRACT

Processes for producing two or more different C₂-C₆ linear or branched olefins are disclosed herein. In one exemplary implementation, the process can include contacting a first feed stream that includes γ-valerolactone with one or more first catalysts in a first reactor to form a mixture. The mixture includes two or more different C₂-C₆ linear or branched olefins at a yield of at least 60%, and the one or more first catalysts include a doped zeolite. Processes for converting levulinic acid to γ-valerolactone are also disclosed herein.

### CROSS-REFERENCE TO RELATED APPLICATION

This application claims priority to U.S. Provisional Patent Application No. 63/398,802 filed on Aug. 17, 2022 and U.S. Provisional Patent Application No. 63/404,465 filed on Sep. 7, 2022, each entitled “Conversion of Levulinic Acid from Celullosic Biomass to Light Olefins,” the disclosures of which are incorporated herein by reference in their entirety.

### TECHNICAL FIELD

The subject matter described herein relates to a processes for producing mixtures of different linear or branched olefins (e.g., ethylene, propylene, butenes, and butadiene).

### BACKGROUND

Approximately 400 million tons of light olefins (ethylene, propylene, butenes, butadiene, etc.) are produced each year for polymer, chemical and pharmaceutical industries. State-of-the-art processes are based on stream cracking of naphtha, and thus rely on fossil fuels, possessing significant environmental impacts. Catalytic production of light olefins from renewable plant biomass is a promising alternative, and many processes have been developed to bridge future gaps in the supply of commodity chemicals from biomass.

Due to decreasing fossil resources, the utilization and development of renewable resources are imminent. As a clean and renewable organic carbon source in nature, biomass is widely used because of its abundant resources and easy availability. Among them, lignocellulose, as the most abundant biomass resource, can be converted into high value-added chemicals, such as levulinic acid (LA), γ-valerolactone (GVL), etc., through thermo-catalytic conversion. GVL may be obtained through various chemical transformations and catalysts, and may be further transformed into chemicals and fuels, such as hydrogenation to obtain 1,4-pentanediol and 2-methyltetrahydrofuran. Additionally, GVL decarboxylation under the action of an acidic catalyst obtains C₄ olefins, which are further subjected to polymerization to obtain C₈⁺ olefins. However, the known techniques in converting LA to GVL are limited in that they produce either butenes or butadienes.

Accordingly, there remains a need for improved catalyst technologies that produce a combination of at least two or more different olefins, which thereby can provide greater flexibility in producing fuels and/or chemicals from bio-based feedstock(s).

### SUMMARY OF THE INVENTION

Aspects of the current subject matter relate to process for producing two or more different C₂-C₆ linear or branched olefins. In some implementations, one or more of the following features may optionally be included in any feasible combination.

In one implementation, an exemplary process for producing two or more different C₂-C₆ linear or branched olefins include contacting a first feed stream that includes γ-valerolactone with one or more first catalysts in a first reactor to form a mixture. The mixture includes two or more different C₂-C₆ linear or branched olefins at a yield of at least 60%, and the one or more first catalysts include a doped zeolite.

In some implementations, the doped zeolite can one or more dopants. In certain implementations, the one or more dopants can include boron, phosphor, or a combination thereof.

In some implementations, the two or more different C₂-C₆ linear or branched olefins can include one or more butenes and butadiene and at least one of ethylene or propylene.

In some implementations, the two or more different C₂-C₆ linear or branched olefins can include one or more butenes. In such implementations, the two or more different C₂-C₆ linear or branched olefins can include butadiene.

In some implementations, the two or more different C₂-C₆ linear or branched olefins can include one or more linear butenes and butadiene.

In some implementations, the first reactor can be at a temperature from about 300° C. to 500° C.

In some implementations, the first reactor can be at a pressure from about 0 psig to 300 psig.

In some implementations, contacting the first feed stream with the one or more first catalysts can include contacting the first feed stream with the one or more first catalysts at a weight hourly space velocity (WHSV) of at least 0.5 to form the mixture.

In some implementations, the process can include contacting a second feed stream that includes levulinic acid with a second catalyst in a second reactor to produce the first feed stream.

In some implementations, the second catalyst can include a mixed metal oxide. In certain implementations, the second catalyst can include ZnZrAlSi. In other implementations, the second catalyst can include ZnZrSi.

In another implementation, an exemplary process for converting γ-valerolactone to two or more different C₂-C₆ linear or branched olefins includes contacting a feed stream that includes γ-valerolactone with one or more catalysts in a reactor at a temperature from about 300° C. to 500° C., a pressure from about 0 psig to 100 psig, and a weight hourly space velocity (WHSV) of at least 1 h⁻¹ to form a mixture. The mixture includes two or more different C₂-C₆ linear or branched olefins at a yield of at least 95%; and the one or more first catalysts includes a zeolite doped with boron and phosphor.

In some implementations, the boron can be present in the doped zeolite in an amount from 1 weight percent to 3 weight percent based on total weight of the doped zeolite. In some implementations, the phosphor can be present in the doped zeolite in an amount from 1 weight percent to 4 weight percent based on total weight of the doped zeolite.

In some implementations, the two or more different C₂-C₆ linear or branched olefins can include ethylene, propylene, butenes, butadiene, or any combination thereof.

In another implementation, an exemplary process for converting levulinic acid to γ-valerolactone includes contacting a feed stream that includes levulinic acid with a catalyst in a reactor to form a mixture. The mixture includes γ-valerolactone at a yield of at least 20%; and the catalyst can include ZnZrAlSi or ZnZrSi.

In some implementations, the mixture can include one or more angelica lactones. In such implementations, the process can include removing the one or more angelica lactones from the mixture.

### BRIEF DESCRIPTION OF THE DRAWINGS

The accompanying drawings, which are incorporated into and constitute a part of this specification, show certain implementations of the subject matter disclosed herein and, together with the description, help explain some of the principles associated with the disclosed implementations. In the drawings:

FIG. 1 is a graph illustrating the product olefin distribution for Examples 2-7.

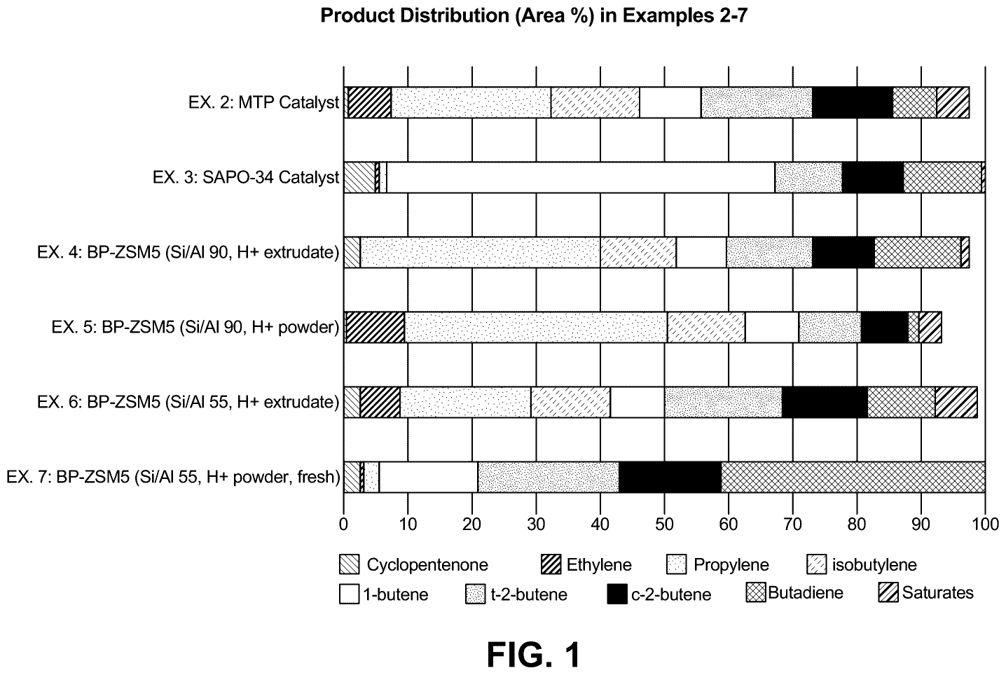

### DETAILED DESCRIPTION

Certain exemplary aspects will now be described to provide an overall understanding of the principles of the structure, function, manufacture, and use of the systems and processes disclosed herein. One or more examples of these aspects are illustrated in the accompanying drawings. Those skilled in the art will understand that the systems and processes specifically described herein and illustrated in the accompanying drawings are non-limiting exemplary aspects and that the scope of the present invention is not defined solely by the claims. The features illustrated or described in connection with one exemplary aspect may be combined with the features of other aspects. Such modifications and variations are intended to be included within the scope of the present invention.

Terminology used herein is for the purpose of describing particular aspects and implementations only and is not intended to be limiting. For example, as used herein, the singular forms “a,” “an,” and “the” are intended to include the plural forms as well, unless the context clearly indicates otherwise.

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

As used herein in the specification and claims, including as used in the examples and unless otherwise expressly specified, all numbers can be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value can have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“WHSV” refers to weight hourly space velocity and is defined as the weight of the feed flowing per unit weight of the catalyst per hour.

“Aromatics” or “aromatic compounds” as used herein refer to cyclic organic carbon compounds consisting of six or more carbons (e.g. benzene, etc.).

All yields and conversions described herein are on a weight basis unless specified otherwise.

The present disclosure generally provides methods for the production of mixtures of at least two different olefins (e.g., ethylene, propylene, butene(s) (e.g., linear or branched butenes), butadiene, and hexene) from GVL. While conventional methods have used GVL as a starting material, these conventional methods do not produce a product stream that is a mixture of different olefins, but instead, for example, the product stream includes either butenes or butadiene. The catalysts described herein allow for the conversion of GVL to a product stream having two or more different olefins. As used herein “different olefins” refers to two or more olefins that differ in number and/or identify of atoms relative to each other. An example of different olefins is 1-butene and 1,3 butadiene because the former has a chemical formula of C₄H₈, and the latter has a chemical formula of C₄H₆. By not different, this means that two or more olefins would have the same chemical formula even if they do not have the same stereochemistry or isomerism. An example of non-different olefins is isobutylene and cis-butene because they both have a chemical formula of C₄H₈.

In general, the present processes include contacting a feed stream (e.g., first feed stream), which includes at least GVL, with one or more catalysts (e.g., one or more first catalysts) in a reactor to form a mixture that includes two or more different C₂-C₆ linear or branched olefins at a yield of at least 60%, in which the one or more catalysts include a doped zeolite. In some implementations, the yield of the two or more different C₂-C₆ linear or branched olefins can be from about 80% to 90%. In other implementations, the yield of the two or more different C₂-C₆ linear or branched olefins can be at least about 98%.

In some implementations, the feed stream can include angelica lactone. In such implementations, the process can include removing the angelica lactone from the feed stream prior to contacting the first feed stream with the one or more first catalysts.

In some implementations, the two or more different C₂-C₆ linear or branched olefins can include one or more butenes and butadiene. In such implementations, the one or more butenes can include linear butenes, branched butenes (e.g., isobutylene), or both. For example, in certain implementations, the two or more different C₂-C₆ linear or branched olefins can include one or more linear butenes and butadiene, whereas in other implementations, the two or more different C₂-C₆ linear or branched olefins can include one or more butenes and butadiene. Alternatively, or in addition, the two or more different C₂-C₆ linear or branched olefins can include ethylene. Alternatively, or in addition, the two or more different C₂-C₆ linear or branched olefins can include propylene. In one implementation, the two or more different C₂-C₆ linear or branched olefins can include one or more linear butenes, isobutylene, butadiene, ethylene, and propylene. It is also contemplated herein that the two or more different C₂-C₆ linear or branched olefins do not include isobutylene, ethylene, propylene, or any combination thereof.

In some implementations where the two or more different C₂-C₆ linear or branched olefins include butadiene, the butadiene can be present in the mixture in amount from about 1 weight percent to 50 weight percent based on total weight of the mixture. In other implementations, the butadiene can be present in the mixture in amount from about 10 weight percent to 45 weight percent based on total weight of the mixture. It is also contemplated that the amount of butadiene present in the mixture does not fall outside any of these recited ranges. It is further contemplated that the amount of butadiene present in the mixture can be between any of these recited ranges.

The one or more catalysts used in the transformation of GVL to the two or more different C₂-C₆ linear or branched olefins can involve, by way of example, the use of one or more doped zeolites. Non-limiting suitable examples of one or more—zeolites can include crystalline silicates of the group ZSM-5 (MFI or BEA frameworks), CHA, FER, FAU, MWW, MOR, EUO, MFS, ZSM-48, MTT, SAPO-34, MTP or TON having Si/Al higher than 10, or a dealuminated crystalline silicate of the group ZSM-5 (MFI or BEA frameworks), CHA, FER, FAU, MWW, MOR, EUO, MFS, ZSM-48, MTT or TON having Si/Al higher than 10.

Suitable dopants of particular usefulness herein for modulating zeolite acidity and hydrothermal stability resulting in desirable yield and selectivity based on GVL conversion, as compared to the undoped H⁺ zeolite form, are phosphor and/or boron together or separately. As such, in some implementations, the one or more doped zeolites can include one or more dopants that include boron, phosphor, or both. In some implementations, the boron can be present in the doped zeolite in an amount from 1 weight percent to 3 weight percent based on total weight of the doped zeolite, and the phosphor can be present in the doped zeolite in an amount from 1 weight percent to 4 weight percent based on total weight of the doped zeolite. In some implementations, the one or more doped zeolites can include a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-alumino-phosphate of the group AEL.

In implementations where the one or more dopants include at least boron, in some implementations, the boron can be present in the doped zeolite in an amount from 1 weight percent to 3 weight percent based on total weight of the doped zeolite. In other implementations, the boron is present in the doped zeolite in an amount from 1.5 weight percent to 2.5 weight percent based on total weight of the doped zeolite. It is also contemplated that the amount of boron present in the one or more dopants does not fall outside any of these recited ranges. It is further contemplated that the amount of boron present in the one or more dopants can be between any of these recited ranges.

In implementations where the one or more dopants include at least phosphor, in some implementations, the phosphor can be present in the doped zeolite in an amount from 1 weight percent to 4 weight percent based on total weight of the doped zeolite. In other implementations, the phosphor can be present in the doped zeolite in an amount from 2 weight percent to 3.5 weight percent based on total weight of the doped zeolite. It is also contemplated that the amount of phosphor present in the one or more dopants does not fall outside any of these recited ranges. It is further contemplated that the amount of boron present in the one or more dopants can be between any of these recited ranges.

In some implementations, the one or more catalysts (e.g., one or more first catalysts) include a boron and phosphor doped ZSM-5 with a Si/Al ratio of 55 or 90.

Additional additives for mixing with doped zeolites can include SiO₂ supports doped with metal dopants. Non-limiting examples of suitable metal dopants can include iron (Fe), strontium (Sr), cobalt (Co), nickel (Ni), lanthanum (La), chromium (Cr), zirconium (Zr), ruthenium (Ru), molybdenum (Mo), iridium (Ir), magnesium (Mg), tungsten (W), copper (Cu), manganese (Mn), vanadium (V) zinc (Zn), titanium (Ti), rhodium (Rh), rhenium (Re), gallium (Ga), palladium (Pd), silver (Ag), indium (In), or any combinations thereof. One or more of the foregoing exemplary metal dopants can be used alternatively or in addition to boron and/or phosphor.

In some implementations, the one or more catalysts described herein can be prepared by incipient wetness impregnation techniques.

In use, the process can be carried out under a variety of temperatures. In some implementations, for example, the temperature of the reactor (e.g., first reactor) can be from 100° C. to 600° C. In other implementations, the temperature of the reactor can be from about 300° C. to 500° C., from about 380° C. to 460° C., or from about 400° C. to 500° C. It is also contemplated that the temperature of the reactor does not fall outside any of these recited ranges. It is further contemplated that the temperature of the reactor can be between any of these recited ranges.

Alternatively, or in addition, the process can be carried under a variety of pressures. In some implementations, for example, the pressure of the reactor (e.g., first reactor) can be from about 0 psig to 500 psig. In other implementations, the pressure of the reactor can be from about 0 psig to 300 psig or from about 0 psig to 100 psig. It is also contemplated that the pressure of the reactor does not fall outside any of these recited ranges. It is further contemplated that the pressure of the reactor can be between any of these recited ranges.

Alternatively, or in addition to, the process can be carried out under a variety of a weight hourly space velocities. In some implementations, for example, the WHSV can be at least 0.5 h⁻¹ or at least 1 h⁻¹. In other implementations, the WHSV can be from about 0.1 h⁻¹ to 15^{h-1}, from about 1^{h-1} to 10^{h-1}, or from about 1^{h-1} to 5^{h-1}. It is also contemplated that the WHSV of the reactor does not fall outside any of these recited ranges. It is further contemplated that the process can be carried out at a WHSV between any of these recited ranges.

In the processes described herein, in certain implementations, the process can be carried out at a temperature from 100° C. to 500° C., a pressure from about 0 psig to 100 psig, and a WHSV of at least 0.5 h⁻¹. In some implementations, a process for converting GVL to two or more C₂-C₆ linear or branched olefins can include contacting a feed stream, which includes γ-valerolactone, with a zeolite doped with boron and phosphor in a reactor at a temperature from about 300° C. to 500° C., a pressure from about 0 psig to 100 psig, and a weight hourly space velocity (WHSV) of at least 1 h⁻¹ to form a mixture, in which the mixture includes two or more different C₂-C₆ linear or branched olefins at a yield of at least 95% and the one or more first catalysts include a zeolite doped with boron and phosphor.

GVL is produced from the conversion of LA. As such, the present processes presented herein can include a method for converting LA to GVL. In general, a process for converting levulinic acid to γ-valerolactone (GVL) can include contacting a feed stream (e.g., a second feed stream), which includes levulinic acid, with a catalyst in a reactor (e.g., a second reactor) to form a mixture that includes GVL. In some implementations, the GVL is produced at a yield of at least 20%, of at least 25%, or of at least 50%. In certain implementations, GVL is produced at a yield from about 25% to 50%, from about 20% to 50%, of about 25% to 40%, or about 25% to 35%. It is also contemplated that the yield of GVL does not fall outside any of these recited ranges. It is further contemplated that the yield of GVL is between any of these recited ranges.

In some implementations, the catalyst can include ZnZrSi or ZnZrAlSi. These catalysts can be prepared via a hard-template method, a co-precipitation method, or an impregnated method, via a co-precipitation method.

For example, in some implementations, the ZnZrSi mixed oxide catalyst can be prepared via the co-precipitation method with carbon black. In one such implementation, precursor metal salts are added to deionized water to produce an appropriate zinc to zirconium ratio. In additional implementations, the zinc and zirconium nitrate mixture may be sonicated to produce a clear solution, or heated to 60° C. until a clear solution is produced. In further additional implementations, the sonicated or heated zinc and zirconium nitrate mixture is added to the flask followed by addition of carbon black. The heterogeneous mixture is stirred for 5-10 minutes to assure complete wetting of carbon black and afterwards the appropriate amount of silicon dioxide is added followed by stirring for an additional 5-10 minutes. The resulting mixture is precipitated, via dropwise addition of 20 weight percent NaOH, LiOH, or KOH, at room temperature with vigorous stirring until a final pH of 6.0-8.0 is attained. Afterwards, the precipitated slurry is allowed to stir at room temperature for an additional 60 minutes. In further additional implementations, the co-precipitated ZnZrSi mixed oxide catalyst may be dried at 140° C., and calcinated at a temperature between 400° C. and 550° C. In an exemplary implementation, calcination occurs at a temperature of 500° C. for a period of 4 hours. In an exemplary implementation, the final ratio of Zn/Zr/Si (x:y:v) in the ZnZrSi mixed oxide catalyst is in a range of about 1:8:1 to about 1:36:4. In a more specific exemplary implementation, the ratio of Zn/Zr/Si (x:y:v) in the ZnZrSi mixed oxide catalyst is about 1:12:2.

For example, in some implementations, the ZnZrAlSi mixed oxide catalyst is prepared using the co-precipitation method with carbon black. In one such implementation, precursor metal salts are added to deionized water to produce an appropriate zinc to zirconium ratio. In additional implementations, the zinc and zirconium nitrate mixture may be sonicated to produce a clear solution, or heated to 60° C. until a clear solution is produced. In further additional implementations, the sonicated or heated zinc and zirconium nitrate mixture is added to the flask followed by addition of finely ground Al₂O₃, SiO₂, and carbon black. The resulting mixture is precipitated, via dropwise addition of 20 weight percent NaOH, at room temperature with vigorous stirring until a final pH of 7.0-8.0 is attained. Afterwards, the precipitated slurry is allowed to stir at room temperature for an additional 60 minutes. In further additional implementations, the co-precipitated ZnZrAlSi mixed oxide catalyst may be dried at 140° C., and calcinated at a temperature between 400° C. and 550° C. In an exemplary implementation, calcination occurs at a temperature of 500° C. for a period of 4 hours. In a specific exemplary implementation, the ratio of Zn/Zr/Al/Si (x:y:v:s) in the ZnZrAlSi mixed oxide catalyst is about 1:12:2:2.

In use, the conversion of LA to GVL can be carried out a variety of temperatures. In some implementations, for example, the reactor (e.g., second reactor) is at a temperature from about 300° C. to 500° C. In other implementations, the temperature of the reactor can be from about 350° C. to 500° C., from about 350° C. to 400° C., or from about 380° C. to 450° C. It is also contemplated that the temperature of the reactor does not fall outside any of these recited ranges. It is further contemplated that the temperature of the reactor can be between any of these recited ranges.

Alternatively, or in addition, the process can be carried under a variety of pressures. In some implementations, for example, the pressure of the reactor (e.g., first reactor) can be from about 0 psig to 500 psig. In other implementations, the pressure of the reactor can be from about 0 psig to 300 psig or from about 0 psig to 100 psig. It is also contemplated that the pressure of the reactor does not fall outside any of these recited ranges. It is further contemplated that the pressure of the reactor can be between any of these recited ranges.

Alternatively, or in addition to, the process can be carried out under a variety of a weight hourly space velocities. In some implementations, for example, the WHSV can be at least 0.5 h⁻¹ or at least 1 h⁻¹. In other implementations, the WHSV can be from about 0.1 h⁻¹ to 10^{h-1}, from about 1^{h-1} to 10^{h-1}, or from about 1^{h-1} to 5^{h-1}. It is also contemplated that the WHSV of the reactor does not fall outside any of these recited ranges. It is further contemplated that the process can be carried out at a WHSV between any of these recited ranges.

In some implementations, in addition to GVL, the conversion of LA can produce one or more angelica lactones. As such, in certain implementations, the mixture can include one or more angelica lactones. The process can also include removing the one or more angelica lactones from the mixture.

It should be noted that the processes described herein for producing two or more C₂-C₆ linear or branched olefins can include the processes described herein for the conversion of LA to GVL, followed by the processes described herein for the conversion of GVL to two or more C₂-C₆ linear or branched olefins. For example, in some implementations, the process for producing two or more C₂-C₆ linear or branched olefins can include contacting a first feed stream that includes levulinic acid with a first catalyst in a reactor to produce a second feed stream, and contacting the second feed stream that includes γ-valerolactone with one or more second catalysts in another reactor to form a mixture that includes two or more different C₂-C₆ linear or branched olefins, in which the one or more first catalysts includes a doped zeolite. In certain implementations, the first catalyst can include ZnZrAlSi or ZnZrSi

The following specific examples are intended to be illustrative and should not be construed as limiting in scope of the claims.

### Examples

Reactor Set-Up: LA conversion to GVL and GVL conversion to olefins was carried out at between 300° C.-500° C., via fixed bed reactors, containing specified catalyst(s), and flowing preheated (160° C.) vaporized feedstock (e.g., LA, water, and formic acid (LA to GVL) or GVL and water (GVL to olefin mixture)) in a downward flow over the fixed catalyst bed while co-feeding nitrogen at atmospheric pressure or under moderate pressures (e.g., 0-30 bar). The feed flow rates were controlled by Teledyne Model 500D syringe pumps, and the flow rates were adjusted to obtain the targeted olefin WHSV (weight hourly space velocity). The internal reaction temperature was maintained constant via a Lindberg Blue M furnace as manufactured by Thermo-Scientific. Conversion and selectivity was calculated by analysis of the liquid phase reactor effluent by GC for organic and water content, and online GC analysis of non-condensed hydrocarbons (e.g., C₂-C₇ olefins) relative to nitrogen as internal standard.

Example 1: LA conversion to AL and GVL—Single Stage reactor configuration: Reaction Conditions: Feed—LA and Formic acid (2 eq relative to LA) in water (36 wt %, 28 wt %, 36 wt %, respectively); T=385° C. in reactor, WHSV=1.0 (LA basis), P=0 bar; Catalyst—Mixed metal oxide Zn/Zr/Al/Si (1/12/2/2 mol ratio); LA conversion=80%

Single Pass Reactor Effluent Composition - Area % of Total:

| | |
|---|---|
| 5-Me-2(3H)-furanone | 27 |
| 5-Me-2(5H)-furanone | 33 |
| GVL | 35 |
| Aromatics | 4 |

Example 2: GVL conversion to olefins—Single Stage reactor configuration: Reaction Conditions: Feed—GVL in water (40 wt %); T=440° C. in reactor, WHSV=3.8 (GVL basis), P=0 bar; Catalyst— MTP commercial catalyst; GVL conversion=83%

Single Pass Reactor Effluent Composition - Area % of Total:

| | |
|---|---|
| 2-cyclopentenone | 0.8 |
| ethylene | 6.5 |
| propylene | 24.8 |
| isobutylene | 14.1 |
| 1-butene | 9.4 |
| butadiene (BD) | 6.8 |
| trans-2-butene | 17.3 |
| cis-2-butene | 12.5 |
| saturates (C₂—C₅) | 5.0 |

Example 3: GVL conversion to olefins—Single Stage reactor configuration: Reaction Conditions: Feed—GVL in water (40 wt %); T=440° C. in reactor, WHSV=3.8 (GVL basis), P=0 bar; Catalyst— SAPO-34 commercial catalyst; GVL conversion=83%

Single Pass Reactor Effluent Composition - Area % of Total:

| | |
|---|---|
| 2-cyclopentenone | 4.8 |
| ethylene | 0.8 |
| propylene | 1.2 |
| isobutylene | 0.0 |
| 1-butene | 60.5 |
| butadiene (BD) | 12.3 |
| trans-2-butene | 10.4 |
| cis-2-butene | 9.6 |
| saturates (C₂—C₅) | 0.5 |

Example 4: GVL conversion to olefins—Single Stage reactor configuration: Reaction Conditions: Feed—GVL in water (40 wt %); T=440° C. in reactor, WHSV=3.8 (GVL basis), P=0 bar; Catalyst— ZSMS H⁺ form zeolite extrudate (Si/Al ratio=90) doped with boron and phosphor; GVL conversion=98.9%

Single Pass Reactor Effluent Composition - Area % of Total:

| | |
|---|---|
| 2-cyclopentenone | 2.6 |
| ethylene | 7.3 |
| propylene | 30.3 |
| isobutylene | 11.7 |
| 1-butene | 7.8 |
| butadiene (BD) | 13.5 |
| trans-2-butene | 13.2 |
| cis-2-butene | 9.6 |
| saturates (C₂—C₅) | 1.5 |

Example 5: GVL conversion to olefins—Single Stage reactor configuration: Reaction Conditions: Feed—GVL in water (40 wt %); T=440° C. in reactor, WHSV=3.8 (GVL basis), P=0 bar; Catalyst— ZSMS H⁺ form zeolite powder (Si/Al ratio=90) doped with boron and phosphor; GVL conversion=99.8%

Single Pass Reactor Effluent Composition - Area % of Total:

| | |
|---|---|
| 2-cyclopentenone | 0.4 |
| ethylene | 9.2 |
| propylene | 40.8 |
| isobutylene | 12.3 |
| 1-butene | 8.2 |
| butadiene (BD) | 1.6 |
| trans-2-butene | 9.9 |
| cis-2-butene | 7.2 |
| saturates (C₂—C₅) | 3.7 |

Example 6: GVL conversion to olefins—Single Stage reactor configuration: Reaction Conditions: Feed—GVL in water (40 wt %); T=440° C. in reactor, WHSV=1.7 (GVL basis), P=0 bar; Catalyst— ZSMS H⁺ form zeolite extrudate (Si/Al ratio=55) doped with boron and phosphor; GVL conversion=99.2%

Single Pass Reactor Effluent Composition - Area % of Total:

| | |
|---|---|
| 2-cyclopentenone | 2.8 |
| ethylene | 5.9 |
| propylene | 20.4 |
| isobutylene | 12.4 |
| 1-butene | 8.2 |
| butadiene (BD) | 10.6 |
| trans-2-butene | 18.6 |
| cis-2-butene | 13.2 |
| C₅ olefins | 3.7 |
| C₂—C₅ saturates | 4.2 |

Example 7: GVL conversion to olefins—Single Stage reactor configuration: Reaction Conditions: Feed—GVL in water (40 wt %); T=440° C. in reactor, WHSV=3.8 (GVL basis), P=0 bar; Catalyst— ZSMS H⁺ form zeolite powder (Si/Al ratio=55) doped with boron and phosphor; GVL conversion=99.8%

Single Pass Reactor Effluent Composition - Area % of Total:

| | |
|---|---|
| 2-cyclopentenone | 2.7 |
| ethylene | 0.4 |
| propylene | 2.5 |
| isobutylene | 0.0 |
| 1-butene | 15.3 |
| butadiene (BD) | 41.3 |
| trans-2-butene | 22.0 |
| cis-2-butene | 15.3 |
| saturates (C₂—C₅) | race |

Although various illustrative aspects are described above, any of a number of changes can be made to various aspects without departing from the teachings herein. For example, the order in which various described method steps are performed may often be changed in alternative aspects, and in other alternative aspects, one or more method steps may be skipped altogether. Optional features of various system and process aspects may be included in some aspects and not in others. Therefore, the foregoing description is provided primarily for exemplary purposes and should not be interpreted to limit the scope of the claims.

The examples and illustrations included herein show, by way of illustration and not of limitation, specific aspects in which the subject matter may be practiced. As mentioned, other aspects may be utilized and derived there from, such that structural and logical substitutions and changes may be made without departing from the scope of this disclosure. Such aspects of the inventive subject matter may be referred to herein individually or collectively by the term “invention” merely for convenience and without intending to voluntarily limit the scope of this application to any single invention or inventive concept, if more than one is, in fact, disclosed. Thus, although specific aspects have been illustrated and described herein, any arrangement calculated to achieve the same purpose may be substituted for the specific aspects shown. This disclosure is intended to cover any and all adaptations or variations of various aspects. Combinations of the above aspects, and other aspects not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other implementations may be within the scope of the following claims.

What is claimed is:

1. A process for producing two or more different C₂-C₆ linear or branched olefins, the process comprising:
contacting a first feed stream comprising γ-valerolactone with one or more first catalysts in a first reactor to form a mixture, the mixture comprising two or more different C₂-C₆ linear or branched olefins at a yield of at least 60%;
wherein the one or more first catalysts comprise a doped zeolite.

2. The process of claim 1, wherein the doped zeolite comprises one or more dopants, the one or more dopants comprising boron, phosphor, or a combination thereof.

3. The process of claim 1, wherein the two or more different C₂-C₆ linear or branched olefins comprise one or more butenes and butadiene and at least one of ethylene or propylene.

4. The process of claim 1, wherein the two or more different C₂-C₆ linear or branched olefins comprise one or more butenes.

5. The process of claim 4, wherein the two or more different C₂-C₆ linear or branched olefins comprises butadiene.

6. The process of claim 1, wherein the two or more different C₂-C₆ linear or branched olefins comprise one or more linear butenes and butadiene.

7. The process of claim 1, wherein the first reactor is at a temperature from about 300° C. to 500° C.

8. The process of claim 1, wherein the first reactor is at a pressure from about 0 psig to 300 psig.

9. The process of claim 1, wherein contacting the first feed stream with the one or more first catalysts comprises contacting the first feed stream with the one or more first catalysts at a weight hourly space velocity (WHSV) of at least 0.5 h⁻¹ to form the mixture.

10. The process of claim 1, further comprising contacting a second feed stream comprising levulinic acid with a second catalyst in a second reactor to produce the first feed stream.

11. The process of claim 10, wherein the second catalyst comprises a mixed metal oxide.

12. The process of claim 10, wherein the second catalyst comprises ZnZrAlSi.

13. The process of claim 10, wherein the second catalyst comprises ZnZrSi.

14. A process for converting γ-valerolactone to two or more different C₂-C₆ linear or branched olefins, the process comprising:
contacting a feed stream comprising γ-valerolactone with one or more catalysts in a reactor at a temperature from about 300° C. to 500° C., a pressure from about 0 psig to 100 psig, and a weight hourly space velocity (WHSV) of at least 1 h⁻¹ to form a mixture, the mixture comprising two or more different C₂-C₆ linear or branched olefins at a yield of at least 95%;
wherein the one or more first catalysts comprise a zeolite doped with boron and phosphor.

15. The process of claim 14, wherein the boron is present in the doped zeolite in an amount from 1 weight percent to 3 weight percent based on total weight of the doped zeolite.

16. The process of claim 14, wherein the phosphor is present in the doped zeolite in an amount from 1 weight percent to 4 weight percent based on total weight of the doped zeolite.

17. The process of claim 14, wherein the two or more different C₂-C₆ linear or branched olefins comprise ethylene, propylene, butenes, butadiene, or any combination thereof.

18. A process for converting levulinic acid to γ-valerolactone, the process comprising:
contacting a feed stream comprising levulinic acid with a catalyst in a reactor to form a mixture, the mixture comprising the γ-valerolactone at a yield of at least 20%;
wherein the catalyst comprises ZnZrAlSi or ZnZrSi.

19. The process of claim 18, wherein the mixture further comprises one or more angelica lactones.

20. The process of claim 19, further comprises removing the one or more angelica lactones from the mixture.
